(19) United States
(12) Patent Application Publication
(10) Pub. No.: US 2009/0011074 A1
(43) Pub. Date: Jan. 8, 2009

(54) Satiety Emulsions And Food Compositions

(76) Inventors: Jadwiga Malgorzata Bialek, Vlaardingen (GB); Sergey Michailovich Melnikov, Vlaardingen (NL); Ingrid Winter, Graz (AT)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/667,777

(22) PCT Filed: Nov. 4, 2005
(86) PCT No.: PCT/EP2005/011909

§ 371 Date: Apr. 18, 2008

(30) Foreign Application Priority Data
Nov. 16, 2004 (EP) 04078133.8
May 2, 2005 (EP) 05076054.5
May 4, 2005 (EP) 05076044.6
Aug. 3, 2005 (EP) 05076804.3

(51) Int. Cl. A61P 3/04; A23D 7/005; A23D 7/01; A23C 9/13; A23L 1/00; A23L 1/035; A23L 2/52

(52) U.S. Cl. 426/2; 426/602; 426/605; 426/590; 426/579; 426/580; 426/589; 426/557; 426/417

(57) ABSTRACT

The present invention provides edible aqueous oil-in-water emulsions comprising 15-70% wt oil having a saturated fatty acid content of at least 20% wt and at least 10% wt of said oil being solid at 37° C., 0.1-5% wt emulsifier which is a protein and 25-84.9% wt water. The oil preferably comprises palm oil and the emulsifier preferably comprises partially denatured egg white. The emulsions provide good satiety benefits and do not adversely affect the taste of products into which they are incorporated. Furthermore the emulsions are very stable which has benefits also for the food products and beverages into which they are incorporated. Also provided are food and beverage products comprising the emulsion of the invention.

### FIELD OF THE INVENTION

The present invention relates to edible emulsions that provide beneficial satiety effects and to food and beverage products comprising these emulsions. These emulsions, foods and beverages are suitable for use in calorie controlled diets and other weight management or weight loss plans.

### BACKGROUND OF THE INVENTION

The incidence of obesity and the number of people considered overweight in countries where a so-called Western diet is adopted has drastically increased over the last decade. Since obesity and being overweight are generally known to be associated with a variety of diseases such as heart disease, hypertension, type 2 diabetes and arthereosclerosis, this increase is a major health concern for the medical world and for individuals alike. Furthermore, being overweight is considered by the majority of the Western population as unattractive.

This has led to an increasing interest by consumers in their health and has created a demand for products that help to reduce or control daily caloric intake and/or control body weight and/or bodily appearance.

Several solutions have been proposed to help individuals to control their body weight. Among these solutions is the use of drugs e.g. to suppress the activity of enzymes in the digestive system. However the use of drugs is generally seen as last resort and is not preferred unless strictly required for medical purposes.

Another proposed solution to the problem of being overweight is to prescribe the individuals concerned a specific diet, for example, a diet with a restricted caloric intake per day. However, a common problem with these diets is that often they do not provide a healthy nutritional balance and/or they are difficult to accommodate in modern lifestyles.

Meal replacer products, such as meal replacement bars and beverages, have also been proposed as part of a healthy diet in order to control or reduce body weight. For example, U.S. Pat. No. 5,688,547 discloses a nutritional meal replacement composition comprising dietary fibre, protein, a cellulose gum and gel.

These meal replacer products are generally ones that are intended to be consumed as a single-serving food product, such as a bar or a drink etc to replace one or two meals per day. The meal replacer products are designed such that on the one hand they provide a restricted caloric intake, but on the other hand they provide a healthy balance of nutritional ingredients and are convenient to incorporate into an individual's daily diet.

However a general problem with meal replacement products and other foods formulated to be consumed as part of a calorie controlled diet (such as low-calorie snack foods) is that whilst these products provide a controlled number of calories per serving and have a good nutritional profile, the consumer of the products feels that the satiety effect obtained from consuming these products is not optimal. This may result in the consumer experiencing hunger feelings in a relatively short time after eating the products, and/or, not feeling fully satiated after eating the products. Both of these scenarios can make it more difficult for a subject to follow a calorie-controlled diet or other weight management plan. The subject may eat additional foods to combat the feelings of hunger and so increase the number of calories consumed, or, they may abandon the diet or plan altogether.

It is well known in the art that food products and beverages with a high fat content tend to provide a feeling of satiety upon consumption. However, high levels of fats in foods and beverages also results in a corresponding increase in the calorie content thereof. Hence, simply increasing the fat content to relatively high levels in order to provide an enhanced satiety benefit is not a realistic proposition for products to be used in a calorie-controlled diet or other weight management plan.

Furthermore, consumers have not shown great willingness to compromise on the taste of their food in order to follow a calorie controlled diet or other weight management plan. Consumers expect their foods to have good taste, be nutritionally balanced and provide good satiety effects in addition to being of a suitable calorie content and being convenient to prepare and consume.

Additionally the food and beverage products need to be physically stable upon storage in order to avoid complaints from the consumer, a possible loss in nutritional quality or a loss of efficacy with respect to their satiety effects.

Therefore, the successful formulator of food or beverage products to be used in a calorie controlled diet, or in a weight management plan, must provide tasty food and beverage products which still have a controlled calorie content and a good nutritional profile and yet at the same time provide good satiety benefits. This is desirable in order to provide the subjects following the diets or plans with the greatest chance of success in loosing or maintaining body weight.

The above problems have been considered by formulators of the above types of food or beverage products, but have not proved easy to overcome.

U.S. Pat. No. 6,517,883 discloses food compositions comprising an oil-in-water emulsion of triglyceride oil which is at least partially solid at ambient to body temperature. The emulsions also comprises a galactolipid based food emulsifier or a phospholipid based emulsifier such as lecithin. The food compositions are said to be capable of giving a prolonged feeling of satiety. However, it has been found that the emulsions have a tendency to be somewhat unstable and generally require special handling as they are susceptible to physical instability upon processing and storage.

EP 477 827 discloses low fat mayonnaise comprising less than 10% wt fat and a protein, the protein may be either egg white and/or whey protein concentrate. Fat free mayonnaise is also disclosed.

However, despite the above developments, there still remains a need to ameliorate one or more of the problems mentioned hereinabove. In particular, there still remains a need to provide food and beverage products which have good taste and which provide good satiety effects to the consumer so that they can be used effectively in calorie controlled diets and other weight management plans. There also still remains a need to provide such food and beverage products and ingredients which exhibit good physical stability and which can be produced using conventional food processing techniques.

In particular, there is a still a need for food and beverage products to be used in calorie controlled diets and other weight management plans which have acceptable taste, calorie content, nutritional profile and which provide good satiety effects. Such products should ideally be convenient to manufacture and should not suffer from unacceptable stability or sensorial problems.

Recognising the demand for tasty, nutritional and convenient food and beverage products that can be used in calorie controlled diets and other weight management plans and yet which provide good satiety effects, research has been carried out to try to address one or more of the problems associated therewith.

Thus the present invention seeks to address one or more of the above-mentioned problems.

### SUMMARY OF THE INVENTION

Surprisingly it has been found that one or more of the above-mentioned problems can be addressed by an oil-in-water emulsion comprising certain fats and certain emulsifiers. These oil-in-water emulsions can conveniently be incorporated into food products and beverages (using conventional food processing techniques) to be used in calorie controlled diets and other weight management plans so providing an enhanced satiety effect thereto. In particular, these oil-in-water emulsions do not detrimentally affect the taste, appearance and/or physical form of the products into which they are incorporated and provide good satiety effects.

Thus according to a first aspect the present invention provides an edible oil-in-water emulsion comprising;

a) 15-70% wt oil having a saturated fatty acid content of at least 20% wt and at least 10% wt of the oil being solid at 37° C.,
b) 0.1-5% wt emulsifier, the emulsifier being a protein; and
c) 25-84.9% wt water.

The emulsions of the invention provide good satiety benefits to the consumer and do not adversely affect the taste, appearance or physical form of food or beverage products into which they are incorporated. Furthermore the emulsifier according to the invention provides for a very stable emulsion which has benefits not only for the emulsion itself but also for the food products and beverages into which it is incorporated. The emulsions are simple to produce and can be manufactured using conventional food processing techniques.

Without wishing to be bound by theory, it is believed that the good stability of the emulsions (processing stability as well as storage stability) is provided by the specific emulsifier used. By using an emulsifier which is a protein, better stability is obtained compared to the use of a lipid or other emulsifier. It is believed that this may occur because the protein emulsifier is able to provide a gelled or cross-linked interface at the oil droplet surface. Moreover, it has been found that a thicker layer is generally formed on the surface of the oil droplet with a protein emulsifier than with a lipid emulsifier and this is believed to aid the stability of the oil droplet. This has advantages for the stability of the emulsion itself and also the food and beverage products into which the emulsion is incorporated as the physical stability of the emulsions may be improved and also possible detrimental effects upon the oxidation of oils in the emulsion and/or food or beverage product into which it is incorporated may be ameliorated. The oil-in-water emulsion stays pourable/spoonable for several weeks even at refrigeration temperatures. A further advantage of providing a stable oil-in-water emulsion is that good satiety effects are obtained for both the emulsion and the food and beverage products into which they are incorporated.

It is preferred that the oil has a saturated fatty acid content of at least 25% wt. The saturated fatty acid content is preferably up to 90% wt. A saturated fatty acid content in the range of from 25% wt to 60% wt has been found to be especially effective, such as 40% wt to 60% wt.

It is preferred that the oil has a monounsaturated fatty acid content of at least 20% wt, more preferably of at least 30% wt. It is preferred that the oil has a polyunsaturated fatty acid content of at least 5% wt.

An especially preferred oil comprises fractionated palm oil.

It is especially preferred that the emulsion comprises 0.3-3% wt emulsifier, most especially one selected from egg and egg derived proteins. Egg white has been found to be a very suitable emulsifier and is especially preferred according to the invention with at least partially denatured egg white being most preferred. A further advantage obtained from the use of egg (white) and egg (white) derived protein emulsifiers is that oxidation of fats both in the emulsion and in the food/beverage composition into which they are incorporated may be reduced, thus increasing stability. This may be caused by low levels of metals that are naturally present in these types of emulsifiers Furthermore, the physical stability of liquid products into which these types of emulsions are incorporated may also be generally improved.

According to a particular embodiment of the invention, the oil-in-water emulsion comprises;

a) 20-60% wt oil comprising fractionated palm oil
b) 0.5-3% wt emulsifier comprising egg or egg derived protein.
c) 40-60% wt water.

According to this embodiment, the oil-in-water emulsion may comprise fractionated palm oil as the only oil, or as part of a blend of oils. If a blend of oils is used, it is preferred that the blend further comprises sunflower oil and/or soy bean oil.

According to a second aspect the present invention provides a food or beverage product comprising the oil-in-water emulsion of the first aspect.

According to a third aspect the present invention provides a method of making a food or beverage product of the second aspect, the method comprising the step contacting the oil-in-water emulsion of the first aspect with the food or beverage product or one or more ingredients thereof to produce the food or beverage product.

According to a fourth aspect the present invention provides the use of an oil-in-water emulsion according to the first aspect or a food or beverage product according to the second aspect, in providing an enhanced feeling of satiety to a person consuming the emulsion and/or to aid adherence to a weight loss or weight control programme and/or in a method of preventing or treating obesity.

According to a fifth aspect the present invention provides a method for inducing satiety in a human or animal, the method comprising the step of administering to a human or animal an oil-in-water emulsion according to the first aspect or a food or beverage product according to the second aspect.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material or conditions of reaction, physical properties of materials and/or use are to be understood as modified by the word “about.” All amounts are by weight, based on the total weight of the relevant product, unless otherwise specified.

By the term “at least partially denatured egg white” as used herein is meant that the egg white has been treated so as to effect at least some degree of denaturisation of the protein, that is that the egg white protein undergoes at least some degree of structural change (especially the secondary structure and above) so that it is no longer completely in its native state. This can be achieved by methods well known in the art, for example, by any of controlled heating, controlled spray drying or controlled mechanical treatment or by any other appropriate method. The person skilled in the art is fully aware of how to detect the presence, and measure the degree of, denaturisation of the protein.

Any of the protein emulsifiers may be at least partially denatured and the above definition would then apply mutatis mutandis.

The term “saturated fatty acid content” as used herein means the weight percentage of saturated fatty acid residues in the oil. For example, a saturated fatty acid content of 50% wt represents an oil wherein 50% wt of the fatty acid residues are saturated fatty acids and 50% wt of the fatty acid residues in total are unsaturated fatty acids (monounsaturated fatty acids and/or polyunsaturated fatty acids).

The term “unsaturated fatty acid content” as used herein means the weight percentage of unsaturated fatty acid residues in the oil. For example, an unsaturated fatty acid content of 40% wt represents an oil wherein 40% wt of the fatty acid residues are unsaturated fatty acids and 60% wt of the fatty acid residues are saturated fatty acids.

The term “monounsaturated fatty acid content” as used herein means the weight percentage of monounsaturated fatty acid residues in the oil. For example, a monounsaturated fatty acid content of 40% wt represents an oil wherein 40% wt of the fatty acid residues are monounsaturated fatty acids and 60% wt of the fatty acid residues in total is saturated and/or polyunsaturated fatty acids.

The term “polyunsaturated fatty acid content” as used herein means the weight percentage of polyunsaturated fatty acid residues in the oil. For example, a polyunsaturated fatty acid content of 10% wt represents an oil wherein 10% wt of the fatty acid residues are polyunsaturated fatty acids and 90% wt of the fatty acid residues in total is saturated and/or monounsaturated fatty acids.

Unless stated otherwise or required by context, the terms “fat” and “oil” are used interchangeably herein.

Unless stated otherwise, all percentages are by weight based on the total weight of the composition.

For a more complete explanation of the above and other features and advantages of the invention, reference should be made to the following description of the preferred embodiments. The preferred embodiments apply to all aspects of the invention and can be used as appropriate for each aspect unless the context requires otherwise.

### DETAILED DESCRIPTION OF THE INVENTION

### (i) Oil

The references herein to “oil” refer to a single oil where only one type of oil is used in the oil-in-water emulsions and to a blend of oils where a blend of at least two different oils is used. The individual oils used in a blend preferably all fulfill the characteristics of the oil described herein. However, one of more of the individual oils used in such a blend may not do so provided that the oil blend has the required characteristics.

The oil-in-water emulsions of the invention comprise 15-70% wt oil having a saturated fatty acid content of at least 20% wt. It is preferred that the oil has a saturated fatty acid content of at least 25% wt, more preferably of at least 30 wt. % and even more preferably of at least 40% wt. most preferably of at least 45% wt, especially at least 50% wt. The oil may be fully saturated but it is preferred that the oil has a saturated fatty acid content of up to 90% wt, more preferably of up to 80% wt, most preferably of up to 70% wt.

It has been found that using an oil having a saturated fatty acid content in the range of from 25% wt or 45% wt to 55% wt or 60% wt is especially effective. This mixture provides a balance of good satiety effects, good stability and nutritional benefits.

It is preferred that the oil-in-water emulsions of the invention comprise 20-60% wt oil having a saturated fatty acid content of at least 20% wt, more preferably 30-55% wt such as 35-50% wt.

Any type of oil, or blend of two or more oils, that has the required saturated fatty acid content according to the invention may be used to produce the oil-in-water emulsions. It is preferred that oils comprising C₁₂-C₂₂ saturated fatty acids are used, especially C₁₄-C₁₈ acids.

Suitable fatty acids which may be present in the oils include, amongst others, C14:0 (myristic acid), C16:0 (palmitic acid) and C18:0 (stearic acid).

Without wishing to be bound by theory, it is believed that oil comprising saturated fatty acid residues is digested more slowly by the body than a monounsaturated or polyunsaturated equivalent. Therefore, by controlling the saturated fatty acid content of the oil, the required satiety effect can be achieved. Furthermore the desired nutritional profile of the emulsion, and thus the food or beverage product into which it is included, can be obtained by controlling the saturated fatty acid content.

The presence of substantial amounts of saturated fatty acids with a carbon chain length of 18 or more in the oil of the present emulsion can have a detrimental effect on the eating quality of the food or beverage product in which it is used. Hence, according to a preferred embodiment, the oil in the present emulsion contains at least 10%, preferably at least 20% of C₁₂-C₁₆ saturated fatty acids. In another preferred embodiment the oil contains not more than 30% wt, preferably not more than 20% wt of saturated fatty acids with a carbon chain length of 18 or more. According to a particularly preferred embodiment, the molar ratio of saturated C₁₈₊ fatty acids to saturated C₁₂-C₁₆ fatty acids in the oil is within the range of 1:1 to 1:10.

As mentioned herein before, the satiety inducing effect of the present emulsion is associated with the presence of substantial amounts of saturated fatty acids, especially palmitic acid. Hence, in particularly preferred embodiment, the oil contains at least 10%, especially at least 20% of palmitic acid.

Whilst the oil used in the oil-in-water emulsions according to the present invention may be fully saturated, it is preferred that the oil further comprises unsaturated fatty acids, either monounsaturated fatty acids and/or polyunsaturated fatty acids. It is especially preferred that the oil comprises both monounsaturated fatty acids and/or polyunsaturated fatty acids in order to provide a more nutritionally desired fatty acid content profile. However, it is important to balance the amount of saturated fatty acids, monounsaturated fatty acids and/or polyunsaturated fatty acids in the oil in order to obtain optimum satiety effects. It is preferred that the oil comprises a greater weight percentage of saturated fatty acids than monounsaturated fatty acids. It is also preferred that the oil comprises a greater weight percentage of saturated fatty acids than polyunsaturated fatty acids. It is especially preferred that the oil comprises at least 50% wt saturated fatty acids.

It is preferred according to the present invention that the oil has an unsaturated fatty acid content of at least 25% wt, more preferably of at least 40% wt. It is also preferred that the oil has an unsaturated fatty acid content of up to 60% wt, more preferably of up to 50% wt, most preferably of up to 40% wt.

Any type of oil that has the required unsaturated fatty acid content according to the invention may be used to produce the oil-in-water emulsions. It is preferred that oils comprising C₁₂-C₂₂ unsaturated fatty acids are used, especially C₁₄-C₁₈ acids.

It is preferred according to the present invention that the oil has a monounsaturated fatty acid content of at least 20% wt, more preferably of at least 30% wt. It is also preferred that the oil has a monounsaturated fatty acid content of up to 60% wt, more preferably of up to 50% wt, most preferably of up to 40% wt.

Preferred monounsaturated fatty acids which may be present in the oils include, amongst others, C14:1 (myristoleic acid), C16:1 (palmitoleic acid), and C18:1 (oleic acid).

It is preferred according to the present invention that the oil has a polyunsaturated fatty acid content of at least 5% wt, more preferably of at least 10% wt. It is also preferred that the oil has a polyunsaturated fatty acid content of up to 40% wt, more preferably of up to 30% wt, most preferably of up to 20% wt.

Preferred polyunsaturated fatty acids which may be present in the oils include, amongst others, C18:2 linoleic acid and C18:3 (alpha-linoleic acid).

It is preferred that the oil is at least partially solid at 20° C. It is further preferred that the oil is also at least partially solid at 37° C. It is preferred that 15% wt or more of the oil is solid at 20° C., preferably 20% wt or more, most preferably 30% wt or more. It is also preferred that 5% wt or more of the oil is solid at 37° C., preferably 10% wt or more. The percentage of the oil which is solid at the given temperatures can be determined by DSC measurements (scanning at a heating rate of 10° C. per minute) to plot the melting behavior of the oil over a given temperature range. The percentage of oil which is molten at a given temperature is calculated by integrating the area under the heating curve.

The oil used according to the present invention may be any oil which comprises the required amounts of saturated fatty acids. Preferably the oil will also comprise the amounts of unsaturated fatty acids (monounsaturated fatty acids and/or polyunsaturated fatty acids) given herein. A preferred oil according to the present invention is fractionated palm oil. Other suitable types include mixtures of plant and dairy based oils provided that they comprise the required amounts of fatty acids. The oil may also be a specially prepared blend of fatty acids in given proportions, such proportions not being found in naturally occurring oils. Suitable oils which may be used, provided that the meet the requirements of the saturated fatty acid content according to the invention, include those that are liquid at ambient temperature e.g. avocado, mustard, coconut cottonseed, fish, flaxseed, grape, olive, palm, peanut, rapeseed (canola oil which is un-hydrogenated rapeseed oil), safflower, sesame, soybean, sunflower and the like and mixtures thereof, and those which are solid at ambient temperature e.g. butter fat, chocolate fat, chicken fat, coconut oil, hydrogenated palm kernel oil, and the like and mixtures thereof. Hardened (hydrogenated) oils, such as hydrogenated soybean may also be used. This list is not exhaustive and the determinant factor for the suitability of an oil, or a blend of two or more oils, according to the invention is whether or not it meets the saturated fatty acid content requirements. Palm oil (fractionated) is especially preferred according to the present invention, especially in an amount of from 15-60% wt, more preferably of from 20-40% wt.

According to one embodiment of the present invention, a mixture of (fractionated) palm oil and sunflower oil is used. If such a mixture is used, it is preferred that the weight ratio of palm oil to sunflower oil is in the range of from 5:1 to 1:5, more preferred 2:1 to 1:2, such as 1:1.

### (ii) Emulsifier

The oil-in-water emulsions comprise 0.1-5% wt emulsifier, the emulsifier being a protein.

Any suitable protein which acts as an emulsifier may be used according to the present invention although it is preferred that the emulsifier is selected from milk and milk derived proteins, egg and egg derived proteins, plant or vegetable and plant or vegetable derived proteins, soy and soy derived proteins, and cereal and cereal derived proteins. Particularly suitable protein emulsifiers egg protein, egg derived protein, soy protein, soy derived protein and combinations thereof. Egg proteins and egg derived proteins are the most preferred types of emulsifiers, with both egg yolk and egg white being suitable. Egg white protein is the most preferred type of emulsifier. Mixtures of such protein emulsifiers may also be used. Of the aforementioned types of emulsifiers, egg and egg derived proteins, especially egg white and egg white derived emulsifiers, and soy and soy derived protein emulsifiers, and mixtures thereof, are especially preferred.

It has been found that especially good results are obtained by using at least partially denatured egg white. The egg protein is preferably pasturised and/or desugared. Suitable partially denatured egg white products are commercially available, for example, OVOBEST dried egg white supergel (high pH) available from OVOBEST Eiprodukte GmbH, Neuenkirchen-Vörden, Germany. Spray dried egg white proteins are especially convenient from the point of view of processing.

Thus according to one embodiment of the present invention, egg or egg derived proteins, especially egg white or egg white derived emulsifiers may be used as the sole emulsifier in the oil-in-water emulsion. However, according to further embodiment of the present invention a mixture of two or more of the aforementioned protein emulsifiers may be used. It is preferred that where a mixture of such emulsifiers may be used, the mixture comprises egg or egg derived proteins, especially egg white or egg white derived emulsifiers. An especially preferred emulsifier mixture comprises egg or egg derived proteins, especially egg white or egg white derived emulsifiers and soy or soy derived protein emulsifiers, especially soy protein isolate. If a mixture comprising 1) egg or egg derived proteins, especially egg white or egg white derived emulsifiers and 2) soy or soy derived protein emulsifiers, especially soy protein isolate is used, it is preferred that the weight ratio of 1:2 in the mixture is in the range of from 5:1 to 1:5, more preferably 2:1 to 1:2, most preferably 1.5:1 to 1:1.5, such as 1:1.

The emulsions of the invention preferably comprise 0.3-3% wt emulsifier, more preferably 0.5-3% wt of the emulsifier, most preferably 0.7-2.5% wt of the emulsifier, such as 1-2% wt.

(iii) Water Content

The oil-in-water emulsions of the present invention comprise 25-84.9% wt water, preferably 35-70% wt, most preferably 40-60% wt.

The continuous water phase of the emulsions may also contain other optional ingredients. Examples include, amongst others, thickeners, preservatives, flavours, vitamins, colourants etc. Preferably, the aqueous phase of the present emulsion contains not more than a marginal amount of dissolved/dispersed components (other than the oil and the protein emulsifier), e.g. not more than 3%, preferably not more than 1% by weight of the aqueous phase, most preferably not more than 0.5% by weight of the aqueous phase. The inclusion of substantial amounts of dissolved components in the aqueous phase adversely affects the handling and processability of the emulsion.

### (iv) The Oil-in-Water Emulsion

In accordance with a particularly preferred embodiment, the combination of oil, protein emulsifier and water constitutes a least 80% wt, preferably at least 90% wt of the emulsion. Even more preferably, said combination constitutes at least 95% wt of the emulsion. Most preferably the combination constitutes at least 98% wt of the emulsion. Although, besides oil, protein emulsifier and water, the present emulsion may contain other ingredients, it is preferred to include not more than marginal quantities of these additional ingredients. Thus, it may be ensured that the viscosity of the present emulsion is sufficiently low to enable easy dispersal in the end product, i.e. a food or beverage product.

It is preferred that the emulsion droplets (i.e. those dispersed in the aqueous continuous phase) have a surface weighted mean diameter (D_{3,2}) of 0.3 to 10 microns, preferably 0.5 to 7 microns, such as 1 to 5 microns. The skilled person is well aware of how to determine the particle size of the oil in water emulsions. Suitable apparatus include the Malvern Mastersizer MS20.

The viscosity of the emulsion will vary according to the formulation and the processing conditions. By selecting the appropriate formulation of the emulsion and the processing conditions to prepare it, an emulsion of the desired viscosity can be prepared. For example, if the emulsion is to be incorporated into a beverage product it may be desirable for it to have a lower viscosity than if it is to be incorporated into a nutrition bar. The person skilled in the art would be fully capable of selecting an appropriate viscosity for the emulsion depending upon the type of food or beverage product into which it is to be incorporated.

Generally the viscosity of the emulsion will increase as the content of oil increases and is also affected by the processing method used. Typically, the viscosity of the present emulsion does not exceed 2 Pa·s at 50 s⁻¹. Preferably the viscosity does not exceed 1 Pa·s at 50 s⁻¹. The viscosity of the present emulsion is suitably determined with a viscometer of the type Carimed™ CSL 50.

According to an especially preferred embodiment of the present invention, the oil-in-water emulsion comprises 20-60% wt oil having a saturated fatty acid content of at least 20% wt, 0.5-3% wt emulsifier which is a protein and 40-60% wt water. According to this embodiment the oil is preferably fractionated Palm oil and the emulsifier is preferably egg or egg derived protein, most preferably at least partially denatured egg white.

These emulsions have been found to give especially good satiety effects, are stable and are of an acceptable taste.

### (v) Preparation of the Emulsion

The oil-in-water emulsions of the present invention may be produced by any appropriate method. Suitable methods of producing the emulsions are well known to those skilled in the art.

An especially suitable method of producing the emulsion has been found to be to melt the oil and to slowly add thereto the emulsifier under conditions of high shear whilst maintaining the oil in the molten state. This forms a dispersion of the emulsifier in the oil which is then slowly added to water (which has been heated to at least the melting temperature of the oil) with mixing under conditions of high shear. The mixture is then homogenised. If a lower homogenisation pressure is used for a given emulsion formulation, e.g. in the range of from 60-100 bar, a liquid emulsion is obtained and if a higher pressure, e.g. above 150 bar is used, a thicker, spoonable emulsion is obtained. The exact pressure to be used will depend upon the formulation of the emulsion and the desired viscosity thereof.

Alternatively, the emulsion may be produced by altering the above method so that the emulsifier is first dissolved in the water and the oil is then added thereto.

(vi) Incorporation into Food and Beverage Products

The oil-in-water emulsions of the present invention may be incorporated into food and beverage products by any suitable method. For example, the emulsion may be contacted by any suitable means with the food or beverage product or one or more ingredients thereof to produce the food or beverage product. It is preferred that the oil-in-water emulsions are simply mixed into the food or beverage product (usually by an electric mixer), although obviously this will depend upon the physical form of the food product. Where the type of food product does not allow for this, e.g. because the food product is a solid or substantially solid product such as a nutrition bar or a pasta product, the emulsion may be mixed with one or more of the food product ingredients prior to producing the food product.

A preferred embodiment of the present method of preparing a food or beverage product, comprises incorporating into said food or beverage from 0.5-10% wt, preferably 1-8% wt of an emulsion as defined herein before. The food product is suitably selected from nutrition bars, pasta products and other cereal products, meal replacement products, puddings, dairy products (e.g. chilled dairy products), alternatives to dairy products (e.g. based on plant derived ingredients), custards, puddings, ice cream, sauces and dressings, fillings, spreads, dips, and breakfast-type cereal products.

The present emulsion is particularly suitable for use in low caloric products. The satiety enhancing function of the present emulsion will allow the preparation of low caloric products that can effectively be used in a weight management program or in the treatment or prevention of obesity. Typically, these low caloric food or beverage products have a calorie content of less than 300 kcal per serving, more preferably they have a calorie content in the range of 50-250 kcal per serving.

(vii) Types of Food and Beverage Products

The present invention further provides a food or beverage product comprising the oil-in-water emulsion of the invention. These products preferably comprise the oil-in-water emulsion of the invention in an amount of from 0.5 to 10% wt, based on the weight of the product, preferably 1 to 8% wt, more preferably 2 to 5% wt. The amount of the emulsion in the food or beverage product will depend upon the product format and is easily determined by the person skilled in the art.

The food product according to the invention may be any food product. It is preferred that the food is selected from nutrition bars, pasta products and other cereal products, meal replacement products, puddings such as mousses and other desserts including frozen confectionery (such as ice-cream, water ices, sorbets and frozen yoghurts), chilled dairy products such as yoghurt, quark and cheese, custards, rice or other similar puddings sauces and dressings such as salad dressings and mayonnaise, fillings, spreads, dips, and breakfast type cereal products such as porridge.

The terms “meal replacer” or “meal replacement products” as used herein also include compositions which are eaten as part of a meal replacement weight loss or weight control plan, for example snack products which are not intended to replace a whole meal by themselves but which may be used with other such products to replace a meal or which are otherwise intended to be used in the plan; these latter products typically have a calorie content in the range of from 50-250 kilocalories per serving.

Meal replacers are generally used by consumers following a calorie controlled diet and are especially preferred food composition according to the invention. They have been found to be especially suitable as they can provide good satiety effects combined with restricted calorie content in a convenient form.

Other food compositions intended to be used as part of a weight loss or weight control plan typically have fewer calories per serving (or per 100 g of product) than their ‘non-diet’ equivalents. The calorie content of these foods is deliberately restricted accordingly. Examples include the so-called low-calorie options of every day foods. Meal replacer composition do not generally fall in this category as there may be no ‘full calorie equivalent’ product and also it is necessary to provide a reasonable number of calories per meal replaced.

The beverage product according to the invention may be any beverage product. Preferred types of beverages include powdered beverages, ready-to-drink beverages and soups. Such beverages may be dairy based, such as milk or yoghurt drinks, or may be soy based drinks. The beverages may also be non-protein containing drinks such as sports-type beverages, tea based beverages, coffee based beverages and fruit or vegetable based beverages. Typically a beverage will contain up to 5% wt or 7% wt of the emulsion of the invention.

(viii) Uses

The oil-in-water emulsions of the present invention and the food and beverage products comprising them are intended for use as part of a calorie controlled diet or other weight loss or weight control programme.

The incorporation of the emulsions into food and beverage products provides an enhanced satiety effect thereto thus making them suitable for use in a calorie controlled diet or other weight loss or weight control programme.

Consuming an oil-in-water emulsion or a food or beverage product according to the invention is intended to enhance and/or prolong the feeling of satiety for the consumer and/or extend the time interval between meals and/or reduce the amount of calories consumed in the following meal. This in turn aids the individual concerned to better adhere to a weight loss or weight control programme.

The consumption of a composition according to the invention may occur as a part of a dietary programme, such as those to reduce or control body weight.

The products or emulsion of the present invention may be consumed as desired, preferably at least daily in order to provide advantageous satiety effects, more preferably at least twice daily.

The products or emulsion of the invention may be consumed by a human or an animal in connection with any one or more of the following; the treatment or prevention of obesity or being overweight; to improve or maintain the perception of body image; aiding compliance with a dietary plan e.g. to control, reduce or maintain body weight, including maintenance of desired body weight following previous weight loss; to extend the time elapsed between taking meals; to control, maintain or reduce daily calorie intake; to suppress appetite. The subject following that plan may be thus better able to reduce, control or maintain their body weight, e.g. by following the dietary plan for a longer period of time and/or adhering more closely to the plan as they feel less temptation to snack or over-eat.

The term “weight loss or weight control programme” as used herein includes regimes, plans and diets followed for controlling body weight and also those followed for medical reasons e.g. to loose weight or to aid other health problems adversely affected by being overweight or obese.

The invention will be further illustrated by reference to the following examples. Further examples within the scope of the invention will be apparent to the person skilled in the art.

### EXAMPLES

### Example 1

### Oil-in-Water Emulsions

Examples of oil-in-water emulsions according to the invention are given in table 1.

TABLE 1

| | % wt | | | | |
|---|---|---|---|---|---|
| Ingredient | 1a | 1b | 1c | 1d | 1e |
| Palm oil | 20.0 | 30.0 | 40.0 | 40.0 | 40.0 |
| (fractionated) | | | | | |
| Partially denatured | 1.0 | 1.0 | 1.0 | 1.5 | 2.0 |
| egg white*¹ | | | | | |
| Water | 79.0 | 69.0 | 59.0 | 58.5 | 58.0 |

*¹Spray dried egg white product, OVOBEST dried egg white supergel (high pH) available from OVOBEST Eiprodukte GmbH, Neuenkirchen-Vörden, Germany.

The fatty acid residue content distribution of the fractionated palm oil is given in Table 2;

TABLE 2

| Type of Fatty Acid | % by weight |
|---|---|
| Saturated | 51 |
| Monounsaturated | 40 |
| Polyunsaturated | 9 |

The fatty acid residue comprises (based on the percentage by weight of the fractionated palm oil); saturated fatty acids (about 44% wt C16 acids, about 5% wt C18 acids and about 2% wt of other saturated acids), monounsaturated fatty acids (about 39% wt C18 acids, about 1% wt of other monounsaturated acids), polyunsaturated fatty acids (about 9% wt C18 acids.

The fractionated palm oil melting behaviour was determined by DSC measurements over the temperature range of from 5° C. to 80° C. The curve showed that the oil is composed of three major fractions with melting temperatures of approximately 12° C., 33° C., and 42° C. Melting was completed at approximately 47° C. The curve showed, that approximately 36% of the palm oil is solid at room temperature (20° C.), and approximately 14% is solid at body temperature (37° C.).

The emulsions were prepared according to the following method; The fractionated Palm oil was melted at 60° C. and subsequently kept at 60° C. in a water bath. The dried partially denatured egg white powder was slowly added to the liquid palm oil under high shear mixing in a Silverson mixer model L4R supplied with a head screen (diameter of holes in a screen ca. 1.5-2.0 mm). Temperature during mixing was kept at around 55° C.

Tap water was heated to 60° C. (140 F) prior to the processing.

The palm oil/egg white dispersion was slowly added (poured) to the tap water under high shear mixing (Silverson at about 7500 rpm for about 2 minutes). The temperature during mixing was kept at 55° C.

The palm oil/egg white emulsion was further processed with a high-pressure homogenizer (HPH) Panda (NS1001L, Niro Soavi which was supplied a jacketed tank was set to 50° C. and controlled with a water bath) at 60 bar using one processing cycle. Pressures in the range of 60-100 Bar resulted in liquid emulsions, while pressures above 150 Bar led to the formation of a more consistent “spoonable” emulsion. The temperature of The resulting oil in water emulsion was filled into sterilised glass jars. To ensure the microbiological stability of the emulsion it was pasteurised at 75° C. for at least 2 minutes at the coldest point in the sample. The glass jars were quenched at −20° C. until they reached storage temperature (ca. 60 minutes for 370 ml glass jars) and then stored at 4-5° C.

### Example 2

### Incorporation into a Food Product

Emulsion 1d of example 1 (7.5 grams) was added to a yoghurt beverage having the composition as given in Table 3 (192.5 grams) to produce a beverage according to the invention. The emulsion was added to the yoghurt beverage with mechanical stirring and the beverage was stirred until the emulsion was well distributed throughout.

TABLE 3
Example 2

| | | % by weight |
|---|---|---|
| | % by weight | of example 2 |
| | of example 2 | (yoghurt |
| | (yoghurt | beverage + |
| Ingredient | beverage) | emulsion) |
| Skimmed milk | 53.56 | 51.55 |
| Whey (liquid) | 28.62 | 27.54 |
| Esprion 300*² | 4.56 | 4.39 |
| Promilk 490*³ | 3.55 | 3.41 |
| Inulin | 0.66 | 0.64 |
| Oligofructose | 0.73 | 0.7 |
| Fermenting culture | 0.02 | 0.02 |
| Example 1d emulsion | — | 3.75 |
| Fruit puree | 8.3 | 8 |
| Total | 100.0% | 100.0% |

*²whey powder containing 28% wt protein and 2% wt fat, available from DMV, The Netherlands
*³milk protein powder containing 48% wt protein and 2% wt fat available from Ingredia, France

The resulting beverage comprised 3.75% wt of the oil-in-water emulsion and 96.25% wt of the yoghurt beverage. The formulation of this beverage after the addition of the oil-in-water emulsion is also given above in Table 3.

A control formulation having the formulation as given in Table 4 was prepared using the same method. This beverage had the same total amount of fat as the yoghurt beverage with the emulsion in Table 3 but did not contain any of the emulsion according to the present invention.

TABLE 4
Control Example A

| Ingredient | % by weight |
|---|---|
| Skimmed milk | 52.2 |
| Cream | 2.17 |
| Whey (liquid) | 27.6 |
| Esprion 300*² | 4.4 |
| Promilk 490*³ | 3.42 |
| Inulin | 0.64 |
| Oligofructose | 0.7 |
| Mazola oil | 0.85 |
| Fermenting culture | 0.02 |
| Fruit puree | 8.0 |
| Total | 100.0% |

The yoghurt beverage of example 2, prior to the addition of the emulsion, had a good taste and this was not detrimentally affected by the addition of the emulsion according to the invention. Also, the beverage was stable upon storage and did not suffer from any problems in appearance and physical from the addition of the emulsion. Furthermore the beverage provided good satiety effects upon consumption, better than the Control Example A.

The formulation comprising the emulsion of the invention in table 3 and the control formulation in table 4 (which did not contain any of the emulsion of the invention but which did contain the same amount of total fat) were both tested for energy intake at subsequent ad lib meals in a clinical study. There were 23 subjects in the trial; 8 men and 15 women. The ages of the subjects were in the range 18-28 and they had a body mass index (BMI) in the range 22.6+/−3.6 kg/m².

The subjects in the trial each consumed 325 ml of one of the two aforementioned formulations at 09.00, the formulation type to be consumed being randomly assigned to the subjects but with approximately the same number of subjects consuming each formulation type. The subjects then answered questions on satiety feelings and fatigue on the hour starting at 09.00 every hour until 17.30. The subjects also answered a questions on gastrointestinal complaints at 09.00 after consuming the formulation, and again each two hours thereafter. The subjects also answered questions on taste and liking of the formulations at 09.15.

The subjects ate an ad lib lunch at 13.00 and an ad lib dinner at 17.00. The energy intake for each subject was measured at both ad lib mealtimes.

The results of the energy intake measurements of the subjects for the ad lib lunch at 13.00 showed; 1) the average energy intake in KJ, of the subjects consuming the formulation comprising the emulsion of the invention was approximately 3690 KJ, and 2) the average energy intake in KJ, of the subjects consuming the control formulation was approximately 3880 KJ. Basing the statistics on a one-tailed analysis, the subjects consuming the formulation comprising the emulsion of the invention consumed on average 5% fewer calories at the ad lib lunch than did the subjects consuming the control formulation. For the total energy intake over the study day (including the ad lib lunch and the ad lib dinner), the subjects consuming the formulation comprising the emulsion of the invention consumed on average 2.6% fewer calories than did the subjects consuming the control formulation.

The above results demonstrate the beneficial effects of the emulsions of the present invention in reducing energy intake at a subsequent meal. They also provide a useful way of reducing calorie intake over the whole day, even if consumed once a day.

### Example 3

### Oil-in-Water Emulsions

A further example of an oil-in-water emulsions according to the invention are given in table 5. This time a blend of fractionated palm oil and sunflower oil and two types of protein emulsifiers (partially denatured egg white and soy protein isolate) are used.

TABLE 5
oil-in-water emulsion of example 3

| Ingredient | % wt |
|---|---|
| Sunflower oil | 20.0 |
| Palm oil | 20.0 |
| (fractionated) | |
| Soy protein isolate*² | 0.75 |
| Partially denatured | 0.75 |
| egg white*¹ | |
| Water | 58.5 |

*¹Spray dried egg white product, OVOBEST dried egg white supergel (high pH) available from OVOBEST Eiprodukte GmbH, Neuenkirchen-Vörden, Germany.
*²Prolisse 500 ex Solae, St Louis, USA.

The fatty acid residue content distribution of the fractionated palm oil is given above in Table 2. The fatty acid residue content distribution of the sunflower oil is given above in Table 6 below. Table 7 gives the fatty acid residue content distribution of the mixture of fractionated palm oil and sunflower oil used in Example 3.

TABLE 6

| Type of Fatty Acid | % by weight |
|---|---|
| Saturated | 11% wt |
| Monounsaturated | 20% wt |
| Polyunsaturated | 69% wt |

The sunflower oil was substantially liquid at room temperature.

TABLE 7

| Type of Fatty Acid | % by weight |
|---|---|
| Saturated | 31% wt |
| Monounsaturated | 30% wt |
| Polyunsaturated | 39% wt |

The emulsion of example 3 was prepared according to the following method;

The fractionated Palm oil was melted at 60° C. and subsequently kept at 60° C. in a water bath. Sunflower oil at approximately 55° C. was added thereto. The dried partially denatured egg white powder and soy protein isolate was slowly added to the oil blend under high shear mixing in a Silverson mixer model L4R supplied with a head screen (diameter of holes in a screen ca. 1.5-2.0 mm). Temperature during mixing was kept at around 55° C.

Tap water was heated to 60° C. (140 F) prior to the processing.

The dispersion obtained above was slowly added (poured) to the tap water under high shear mixing (Silverson at about 7500 rpm for about 2 minutes). The temperature during mixing was kept at 55° C.

The emulsion obtained above was further processed with a high-pressure homogenizer (HPH) Panda (NS10001L, Niro Soavi which was supplied a jacketed tank was set to 50° C. and controlled with a water bath) at 60 bar using one processing cycle. Pressures in the range of 60-100 Bar resulted in liquid emulsions, while pressures above 150 Bar led to the formation of a more consistent “spoonable” emulsion. The resulting oil in water emulsion was filled into sterilised glass jars. To ensure the microbiological stability of the emulsion it was pasteurised at 75° C. for at least 2 minutes at the coldest point in the sample. The glass jars were quenched at −20° C. until they reached storage temperature (ca. 60 minutes for 370 ml glass jars) and then stored at 4-5° C.

1. An edible aqueous oil-in-water emulsion comprising:
a) 15-70% wt oil having a saturated fatty acid content of at least 20% wt and at least 10% wt of the oil being solid at 37° C.,
b) 0.1-5% wt emulsifier, the emulsifier being a protein selected from the group consisting of egg protein, egg derived protein, soy protein, soy derived protein and combinations thereof; and
c) 25-84.9% wt water;
the oil, the emulsifier and the water together constituting a least 80% wt of the emulsion and the viscosity of said emulsion not exceeding 2 Pa·s at 50 s⁻¹.

2. An emulsion according to claim 1, wherein the oil, the emulsifier and the water together constitute at least 90% wt of the emulsion.

3. An emulsion according to claim 1, wherein the oil contains at least 10%, preferably at least 20% of C₁₂-C₁₆ saturated fatty acids.

4. An emulsion according to claim 3, wherein the oil contains at least 10%, preferably at least 20% of palmitic acid.

5. An emulsion according to claim 1, wherein the oil contains not more than 30% wt, preferably not more than 20% wt of saturated fatty acids with a carbon chain length of 18 or more.

6. An emulsion according to claim 1, wherein the emulsion comprises 25-60% wt oil.

7. An emulsion according to claim 1, wherein the oil has a saturated fatty acid content of at least 30% wt.

8. An emulsion according to claim 7, wherein the oil has a saturated fatty acid content of at least 40% wt.

9. An emulsion according to claim 1, wherein the oil has a saturated fatty acid content of up to 90% wt.

10. An emulsion according to claim 1, wherein the oil has a monounsaturated fatty acid content of at least 20% wt.

11. An emulsion according to claim 10, wherein the oil has a monounsaturated fatty acid content of at least 30% wt.

12. An emulsion according to claim 1, wherein the oil has a polyunsaturated fatty acid content of not more than 40% wt, preferably of not more than 30% wt.

13. An emulsion according to claim 1, wherein the oil comprises fractionated palm oil.

14. An emulsion according to claim 1, wherein the emulsion comprises 0.3-3% wt emulsifier.

15. An emulsion according to claim 1, wherein the emulsifier comprises egg white.

16. An emulsion according to claim 15, wherein the emulsifier comprises at least partially denatured egg white.

17. An emulsion according to claim 1, wherein the emulsion comprises 35-70% wt water.

18. An emulsion according to claim 1, wherein the emulsion comprises;
a) 20-60% wt oil
b) 0.5-3% wt emulsifier
c) 40-60% wt water.

19. An emulsion according to claim 1, wherein the oil droplets dispersed in the aqueous continuous phase have a surface weighted mean diameter (_{D3,2}) of 0.3 to 10 microns.

20. A method of preparing a food or beverage product, said method comprising incorporating into said food or beverage from 0.5-10% wt, preferably 1-8% wt of an edible aqueous oil-in-water emulsion comprising;
a) 15-70% wt oil having a saturated fatty acid content of at least 20% wt and at least 10% wt of the oil being solid at 37° C.,
b) 0.1-5% wt emulsifier, the emulsifier being a protein; and
c) 25-84.9% wt water.

21. A method according to claim 20, wherein the food is selected from nutrition bars, pasta products and other cereal products, meal replacement products, puddings, dairy products, dairy alternatives, ice cream, custards, puddings, sauces and dressings, fillings, spreads, dips, and breakfast-type cereal products.

22. A method according to claim 20, wherein the food or beverage product has a calorie content in the range of 50-250 kcal per serving.

23. A food or beverage product comprising 0.5-10 wt. % of the oil-in-water emulsion of claim 1.

24. A food or beverage product according to claim 23, wherein the food or beverage product comprises 0.75% to 5% wt oil provided by the oil-in-water emulsion.

25. A food product according to claim 23, wherein the food product is selected from nutrition bars, pasta products and other cereal products, meal replacement products, puddings, dairy products, dairy alternatives, ice cream, custards, puddings, sauces and dressings, fillings, spreads, dips, and breakfast-type cereal products.

26. A beverage product according to claim 23, wherein the beverage is selected from powdered beverages, ready-to-drink beverages and soups.

27. The use of an oil-in-water emulsion in the manufacture of a food or beverage product for use in a method of preventing or treating obesity, said emulsion being an edible aqueous oil-in-water emulsion comprising;
a) 15-70% wt oil having a saturated fatty acid content of at least 20% wt and at least 10% wt of the oil being solid at 37° C.,
b) 0.1-5% wt emulsifier, the emulsifier being a protein; and
c) 25-84.9% wt water.

28. A method for inducing satiety in a human or animal, the method comprising the step of administering to a human or animal a food or beverage product according to claim 23.
